SEQUENCE LISTING (24 sequences)

SEQ ID NO: 1 - DNA, 48 bp, Artificial
  primer PTSG1
tgcccgccgttgtatcgcatgttatggcagggggatcgatcctctaga

SEQ ID NO: 2 - DNA, 45 bp, Artificial
  Primer PTSG2
tgcagcaaccagagccggtgccatttcgctgggccgacaggcttt

SEQ ID NO: 3 - DNA, 48 bp, Artificial
  Primer PTSG3
tgggcgtcggttccgcgaatttcagctggctgcccgccgttgtatcgc

SEQ ID NO: 4 - DNA, 48 bp, Artificial
  Primer PTSG4
gaggttagtaatgttttctttaccaccaaatgcagcaaccagagccgg

SEQ ID NO: 5 - DNA, 48 bp, Artificial
  Primer PYKF1
tggacgctggcatgaacgttatgcgtctgagggtagatttcagtgcaa

SEQ ID NO: 6 - DNA, 48 bp, Artificial
  Primer PYKF2
cgcctttgctcagtaccaactgatgagccggggttccattcaggtcga

SEQ ID NO: 7 - DNA, 48 bp, Artificial
  Primer PYKF3
ccgaatctgaagagatgttagctaaaatgctggacgctggcatgaacg

SEQ ID NO: 8 - DNA, 48 bp, Artificial
  Primer PYKF4
aagtgatctctttaacaagctgcggcacaacgcctttgctcagtacca

SEQ ID NO: 9 - DNA, 48 bp, Artificial
  Primer MQO1
ggcataccatgccggatgtggcgtatcattggggtttaagggcaccaa

SEQ ID NO: 10 - DNA, 48 bp, Artificial
  Primer MQO2
gaactacggcgagatcacccgccagttaatgccccgggctttgcgccg

SEQ ID NO: 11 - DNA, 48 bp, Artificial
  Primer MQO3
tggcgcgtcttatcagcatacgccacatccggcataccatgccggatg

SEQ ID NO: 12 - DNA, 48 bp, Artificial
  Primer MQO4
agccacgcgtacggaaattggtaccgatgtgaactacggcgagatcac

SEQ ID NO: 13 - DNA, 48 bp, Artificial
  Primer SDH1
cagtcagagaatttgatgcagttgtgattgatcgggggggggggaaag

SEQ ID NO: 14 - DNA, 48 bp, Artificial
  Primer SDH2
atcggctctttcaccggatcgacgtgagcgatcccaattctgattaga

SEQ ID NO: 15 - DNA, 48 bp, Artificial
  Primer SDH3
gttgtggtgtggggtgtgtgatgaaattgccagtcagagaatttgatg

SEQ ID NO: 16 - DNA, 48 bp, Artificial
  Primer SDH4
atcatgtagtgacaggttgggataaccggaatcggctctttcaccgga

SEQ ID NO: 17 - DNA, 48 bp, Artificial
  Primer ACEBA1
gcaccttgtgatggtgaacgcaccgaagaacgagctcggtacccgggc

SEQ ID NO: 18 - DNA, 48 bp, Artificial
  Primer ACEBA2
ctttcgcctgttgcagcgcctgaccgccagcaatagaccagttgcaat

SEQ ID NO: 19 - DNA, 48 bp, Artificial
  Primer ACEBA3
gacgcgccgattactgccgatcagctgctggcaccttgtgatggtgaa

SEQ ID NO: 20 - DNA, 48 bp, Artificial
  Primer ACEBA4
atcccgacagatagactgcttcaatacccgctttcgcctgttgcagcg

SEQ ID NO: 21 - DNA, 49 bp, Artificial
  Primer PYKA1
cacctggttgtttcagtcaacggagtattacatcgggggggggggaaag

SEQ ID NO: 22 - DNA, 47 bp, Artificial
  Primer PYKA2
gtggcgttttcgccgcatccggcaacgtacatcccaattctgattag

SEQ ID NO: 23 - DNA, 48 bp, Artificial
  Primer PYKA3
ttatttcattcggatttcatgttcaagcaacacctggttgtttcagtc

SEQ ID NO: 24 - DNA, 49 bp, Artificial
  PrimerPYKA4
gttgaactatcattgaactgtaggccggatgtggcgttttcgccgcatc

(19) United States
(12) Patent Application Publication
(10) Pub. No.: US 2009/0075352 A1
(43) Pub. Date: Mar. 19, 2009

(54) Method For Improving A Strain Based On In-Silico Analysis

(75) Inventors: Sang Yup Lee, Daejeon (KR); Tae Yong Kim, Gyeonggi-do (KR); Dong Yup Lee, Gyeonggi-do (KR); Sang Jun Lee, Daejeon (KR)

Correspondence Address: INTELLECTUAL PROPERTY / TECHNOLOGY LAW, PO BOX 14329, RESEARCH TRIANGLE PARK, NC, 27709, US

(73) Assignee: KOREA ADVANCED INSTITUTE OF SCIENCE AND TECHNOLOGY, Daejeon (KR)

(21) Appl. No.: 11/722,632

(22) PCT Filed: May 23, 2005
(86) PCT No.: PCT/KR2005/001501

§ 371 Date: Sep. 4, 2008

(30) Foreign Application Priority Data
Apr. 8, 2005 (KR) 10-2005-0029568

(51) Int. Cl. C12P 7/46; G06G 7/48; C12N 1/21

(52) U.S. Cl. 435/145; 703/11; 435/252.33

(57) ABSTRACT

The present invention is related to a method for improving a strain on the basis of in silico analysis, in which it compares the genomic information of a target strain for producing a useful substance to the genomic information of a strain overproducing the useful substance so as to primarily screen genes unnecessary for the overproduction of the useful substance, and then to secondarily screen genes to be deleted through performing simulation with metabolic flux analysis. According to the present invention, an improved strain can be effectively constructed by the metabolic and genetic engineering approach comprising comparatively analyzing the genomic information of a target strain for producing a useful substance and the genomic information of a strain producing a large amount of the useful substance to screen candidate genes and performing in silico simulation on the screened candidate genes to select a combination of genes to be deleted, which shows an improvement in the production of the useful substance. Accordingly, the time, effort and cost required for an actual wet test can be significantly reduced.

### TECHNICAL FIELD

The present invention is related to a method for improving a strain on the basis of in silico analysis, in which it compares the genomic information of a target strain for producing a useful substance to the genomic information of a strain overproducing the useful substance so as to primarily screen genes unnecessary for the overproduction of the useful substance, and then to secondarily screen genes to be deleted through performing simulation with metabolic flux analysis

### BACKGROUND ART

Metabolic flux studies provide a variety of information required to alter the metabolic characteristics of cells or strains in the direction we desire, by introducing new metabolic pathways or removing, amplifying or modifying the existing metabolic pathways using molecular biological technology related to the genetic recombinant technology. Such metabolic flux studies include the overall contents of bioengineering, such as the overproduction of existing metabolites, the production of new metabolites, the suppression of production of undesired metabolites, and the utilization of inexpensive substrates. With the aid of increasing bioinformatics newly developed therewith, it became possible to construct each metabolic network model from the genomic information of various species. By the combination of the metabolic network information with the metabolic flux analysis technology, industrial application possibilities for the production of various primary metabolites and useful proteins are now shown (Hong et al., Biotech. Bioeng, 83:854, 2003; US 2002/0168654).

Mathematical models for analyzing cellular metabolism can be divided into two categories, i.e., a model including dynamic and regulatory mechanism information, and a static model considering only the stoichiometric coefficients of biochemical pathways. The dynamic model delineates the dynamic conditions of cells by predicting intracellular changes with time. However, the dynamic model requires many kinetic parameters and thus has a problem in exactly predicting the inner part of cells.

On the other hand, the static mathematical model uses the mass balance of biochemical pathways and cellular composition information to identify an ideal metabolic flux space that available cells can reach. This metabolic flux analysis (MFA) is known to show the ideal metabolic flux of cells and to exactly describe the behavior of cells, even though it does not require dynamic information (Varma et al., Bio-Technol, 12:994, 1994; Nielsen et al., Bioreaction Engineering Principles, Plenum Press, 1994; Lee et al., Metabolic Engineering, Marcel Dekker, 1999).

The metabolic flux analysis is the technology to quantify metabolic fluxes in a organism. The metabolic flux analysis is based on the assumption of a quasi-steady state. Namely, since a change in the concentration of internal metabolites caused by a change in external environment is very immediate, this change is generally neglected and it is assumed that the concentration of internal metabolites is not changed.

If all metabolites, metabolic pathways and the stoichiometric matrix in the pathways (Sᵢⱼ^{T}, metabolite i in the j reaction) are known, the metabolic flux vector (vⱼ, flux of j pathway) can be calculated, in which a change in the metabolite X with time can be expressed as the sum of all metabolic fluxes. Assuming that a change in X with time is constant i.e., under the assumption of the quasi-steady state, the following equation is defined:

S^{T}v=dX/dt=0

However, there are many cases where only pathways are known and stoichiometric value for each metabolite and pathway and the metabolic flux vector (vⱼ) are partially known, and thus, the above equation is expanded to the following equation:

S^{T}v=Sₘvₘ+Sᵤvᵤ=0

The above equation is divided into two matrices; a defined matrix of experimentally known stoichiometric value (Sₘ(I×M), I=total metabolite number, M=total stoichiometrically-known reaction number) times flux (vₘ(M×I)) and a matrix of unknown stoichiometric value (Sᵤ(I×M)) times flux (vᵤ(M×I)). In this regard, m is a subscript for measurement value, and u is a subscript for unmeasurable value.

If the rank (Sᵤ) of the unknown flux vector (Sᵤ) is equal to or greater than u (i.e., if the number of variables is equal to or smaller than an equation), flux is then determined from a simple matrix calculation. However, if the number of variables is greater than an equation (i.e., if a superposed equation exists), operations for verifying the consistency of total equations, accuracy for the measurement values of metabolic flux, and the validity of a quasi-steady state, will be performed for the calculation of more accurate values.

If the number of variables is greater than an equation, the optimal metabolic flux distribution is then calculated by linear programming using specific objective functions and various physicochemical equations where the flux value of a specific metabolic reaction can be limited to a specific range. This can be calculated as follows:
minimize/maximize: Z=Σcᵢvᵢ
s.t. S^{T}v=0 and α_{min,i}≦vᵢ≦α_{max,I}
wherein cᵢ is weighted value, and vᵢ is metabolic flow.

Generally, the maximization of biomass formation rate (i.e., specific growth rate), the maximization of metabolite production and the minimization of byproduct production, and the like, are used as the objective functions. α_{max,i} and α_{min,i} are limit values which each metabolic flux can have, and they can assign the maximum and minimum values permissible in each metabolic flux.

Up to now, various methods for improving strains have been proposed for the highest possible production of useful metabolites, but had a difficulty in improving strains because processes of screening genes and confirming strains with excellent productivity were complicated. The metabolic flux analysis as described above can be used to determine the highest production yield of the desired metabolite by strain improvement, and the determined value can be used to understand the characteristics of metabolic pathways in strains. By determining the characteristics of metabolic pathways, metabolic pathways in need of operations can be determined and a strategy for the operation of metabolic circuits can be established. This makes it possible to control metabolic flux in the most efficient manner and to produce the desired metabolite.

Accordingly, the present inventors have found that a strain can be simply improved by comparing the genomic information on the central metabolic pathways of a target strain for producing a useful substance to the genomic information on the central metabolic pathways of a strain overproducing the useful substance so as to screen genes unnecessary for or interfering with the growth of cells. Subsequently, the present inventors performed the metabolic flux analysis on various combinations of these candidate genes to screen a set of genes that are finally to be deleted, considering both specific growth rate and the formation rate of useful substances, thereby completing the present invention.

### DISCLOSURE OF THE INVENTION

Therefore, it is a main object of the present invention to provide a method for improving a target strain by in silico analysis, in which genomic information and metabolic flux analysis technology are used to improve the target strain for producing a useful substance.

Another object of the present invention is to provide a method for improving a target strain for producing succinic acid by in silico analysis.

Still another object of the present invention is to provide a succinic acid-overproducing mutant strain improved by aformentioned method, as well as a method for preparing succinic acid using the same.

To achieve the above object, in one aspect, the present invention provides a method for improving a useful substance-producing strain, the method comprising the steps of:
(a) selecting a target strain for producing a useful substance and a useful substance-overproducing strain, and constructing metabolic flux analysis model systems for the two strains;
(b) screening genes absent in the useful substance-overproducing strain among genes which are present in the target strain for producing a useful substance and are unnecessary for or interfere with the growth of cells;
(c) constructing combinations of genes to be deleted, from the screened genes;
(d) performing in silico simulation on a mutant strain obtained by deleting each of the combinations of genes constructed in the step (c), from the target strain for producing a useful substance, using the metabolic flux analysis model systems constructed in the step (a);
(e) selecting a combination of genes to be deleted, which is excellent in useful substance production yield versus specific growth rate, from the simulation results; and
(f) constructing a mutant strain with a deletion of the selected combination of genes.

The method for improving the useful substance-producing strain may additionally comprise the step of: (g) culturing the constructed mutant strain to experimentally examine the useful substance production of the mutant strain. Also, the in silico simulation is preferably performed by plotting a trade-off curve between product formation rate and specific growth rate and comparing the specific growth rate of the mutant strain to the yield of the useful substance.

In another aspect, the present invention provides a method for improving a succinic acid-producing strain, the method comprising the steps of:
(a) selecting a target strain for producing succinic acid and a succinic acid-overproducing strain and constructing metabolic flux analysis model systems for the two strains;
(b) screening genes absent in the succinic acid-overproducing strain among genes which are present in the target strain for producing succinic acid and are unnecessary for or interfere with the growth of cells;
(c) constructing combinations of genes to be deleted from the screened genes;
(d) performing in silico simulation on a mutant strain obtained by deleting each of the combinations of genes constructed in the step (c), from the target strain for producing succinic acid, using the metabolic flux analysis model systems constructed in the step (a);
(e) selecting a combination of genes to be deleted, which is excellent in succinic acid production yield versus specific growth rate, from the simulation results; and
(f) constructing a mutant strain with a deletion of the selected combination of genes

In the inventive method for improving the succinic acid-producing strain, the genes screened in the step (b) are preferably selected from the group consisting of ptsG, pykF, pykA, mqo, sdhA, sdhB, sdhC, sdhD, aceB and aceA, and the combination of genes to be deleted, which is selected in the step (e), preferably consists of ptsG, pykF and pykA.

The inventive method for improving the succinic acid-producing strain may additionally comprise the step of: (g) culturing the constructed mutant strain to experimentally examine succinic acid production of the mutant strain. Also, the in silico simulation is preferably performed by plotting a trade-off curve between product formation rate and specific growth rate and comparing the specific growth rate of the mutant strain to the yield of the succinic acid.

In the present invention, the succinic acid-overproducing strain is preferably the genus Mannheimia. The genus Mannheimia strain is preferably Mannheimia succiniciproducens MBEL55E (KCTC 0769BP), and the target strain for producing succinic acid is preferably E. coli.

In still another aspect, the present invention provides a mutant strain with deletions of ptsG, pykF and pykA genes and having the ability to produce high yield of succinic acid, as well as a method for producing succinic acid, comprising culturing the mutant strain in anaerobic conditions. In the present invention, the mutant strain is preferably an E. coli strain with deletions of ptsG, pykF and pykA genes.

Other features and embodiments of the present invention will be more clearly understood from the following detailed description and accompanying claims.

### BRIEF DESCRIPTION OF DRAWINGS

FIG. 1 is a flow chart showing a method for improving a strain according to the present invention.

FIG. 2 shows a method for screening candidate genes to improve a useful substance-producing strain according to the present invention.

FIG. 3 shows a process of screening candidate genes to improve a succinic acid-producing strain according to the present invention and constructing a mutant E. coli strain.

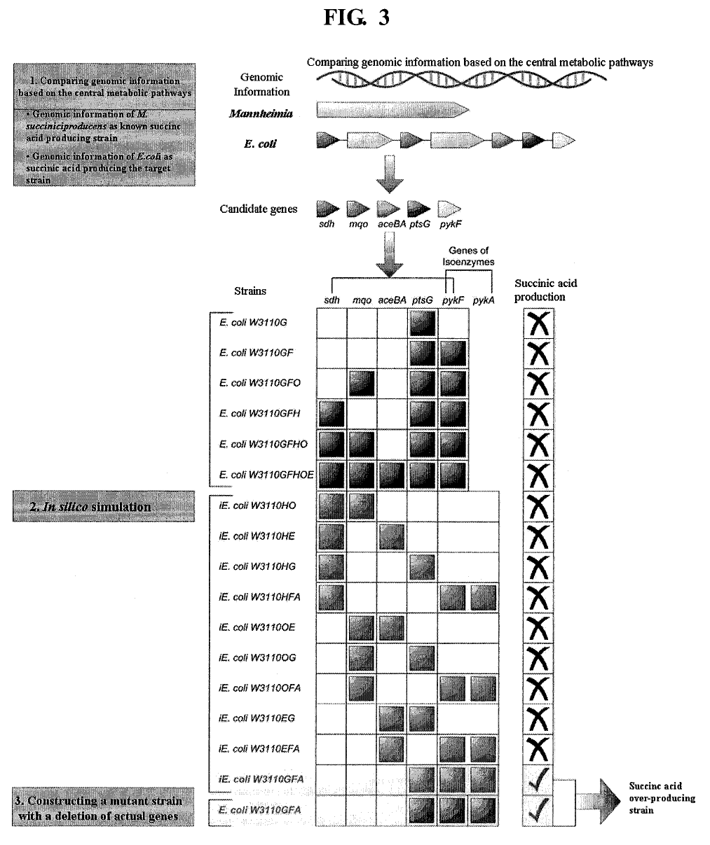

FIG. 4 shows the comparison of metabolic pathways between succinic acid-overproducing strain Mannheimia (A) and a target strain E. coli (B) for producing the useful substance.

FIG. 5a and FIG. 5b shows trade-off curves between succinic acid production and specific growth rate, in which FIG. 5a shows trade-off curves caused by the deletion of one gene (-◯-: ptsG; -▪-: ΔaceBA; -Δ-: wild type/pykFA/sdhA/mqo), and FIG. 5b shows trade-off curves for all possible 10 combinations caused by the deletion of two genes (-◯-: ΔptsGΔpykAF, -▪-: ΔptsGΔmqo/ΔptsGΔsdhA/ΔptsGΔaceBA; -Δ-: ΔpykAFΔmqo/ΔpykAFΔsdhA/ΔpykAFΔaceBA/ΔmqoΔsdhA/ΔmqoΔaceBA/ΔsdhAΔaceBA).

FIG. 6 shows an example of a trade-off curve plotted using MetaFluxNet.

### DETAILED DESCRIPTION OF THE INVENTION, AND PREFERRED EMBODIMENTS THEREOF

In the present invention, the method for improving a strain by screening target genes, which allows the in silico prediction of the results obtained by deleting specific genes to artificially change intracellular metabolic pathways, was developed.

For the improvement of a strain according to the present invention, genes are first screened, which are absent in the useful substance-overproducing strain but present in the target strain for producing a useful substance, and are unnecessary for or interfere with the growth of cells.

Then, one or more combinations of the screened genes are made. Among these gene combinations, a combination of genes is further screened, which shows highly useful substance formation rate versus specific growth rate when the candidate genes were deleted from the target strain for producing a useful substance using a metabolic flux analysis program.

The combination of the secondarily screened genes is deleted from the target strain so as to construct a mutant strain producing the useful substance, and the constructed mutant strain is cultured and examined for the production of the useful substance.

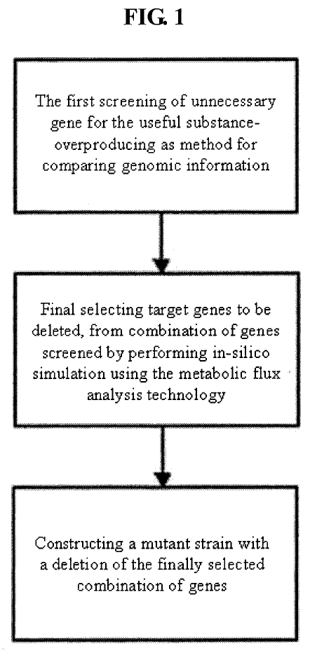

FIG. 1 is a flow chart of the inventive method for selecting a strain for the mass production of succinic acid. As shown in FIG. 1, genes are first screened, which are absent in the useful substance-overproducing strain but present in the target strain for producing the useful substance and are unnecessary for or interfere with the growth of cells. Metabolic flux analysis technology is used to compare the curves of succinic acid production versus specific growth rate, and then, a mutant strain with a deletion of the combination of the candidate genes is constructed.

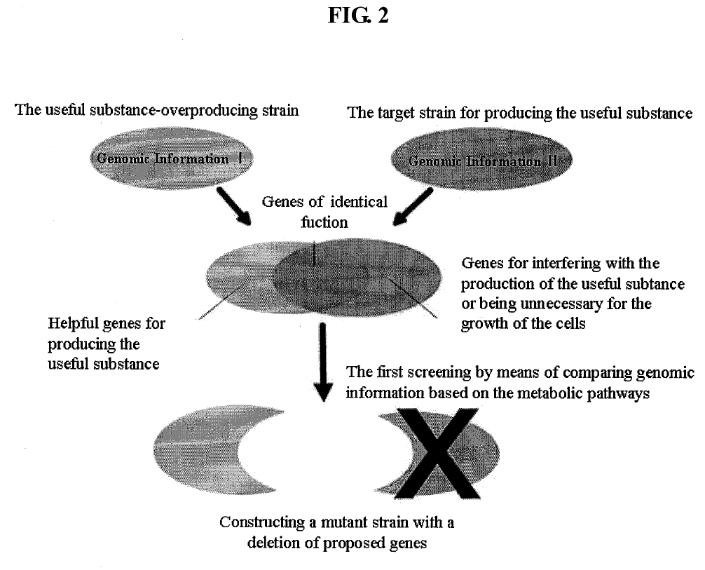

FIG. 2 shows a method for performing the first screening of candidate genes by the use of genomic information to improve a useful substance-producing strain. As shown in FIG. 1, in the first screening, the presence or absence of genes is examined for each strain to screen genes.

In the present invention, genes are first screened, which are absent in the useful substance-overproducing strain but present in the target strain for producing the useful substance and are unnecessary for or interfere with the growth of cells.

The screened genes are deleted from the target strain to make mutations of the target strain for producing the useful substance. The mutations are subjected to in silico simulation, and among them, a mutant strain showing an improvement in the production of the useful substance is selected, and finally examined for the production of the useful substance by actual culture tests.

In the present invention, as model systems for applying the above method, E. coli mutant strain and recombinant E. coli strain were selected and applied to the production of succinic acid.

As used herein, the term “deletions of genes” means to include all operations making specific genes inoperable, including removing or modifying all or a portion of the base sequences of the genes.

### EXAMPLES

Hereinafter, the present invention will be described in more detail by examples. It is to be understood, however, that these examples are for illustrative purposes only and are not construed to limit the scope of the present invention.

Particularly, although the following examples illustrated a method for improving succinic acid-producing strain E. coli by comparison with the genomic information of Mannheimia succiniciproducens, which is a succinic acid-overproducing strain, it will be obvious to a person skilled in the art from the disclosure of the present invention that other succinic acid-overproducing strains and other strains for producing succinic acid can also be used. Moreover, although the following examples illustrated only succinic acid as a useful substance, it will be obvious to a person skilled in the art from the disclosure of the present invention that strains producing other useful strains in addition to succinic acid can be improved according to the present invention.

### Example 1

### Construction of Model systems

As model systems, an E. coli mutant strain, a recombinant E. coli strain and Mannheimia succiniciproducens, which is a succinic acid-producing strain, were selected. For this purpose, new metabolic flux analysis systems for E. coli and Mannheimia were constructed.

(A) E. coli

In the case of E. coli, new metabolic pathway consisted of 979 biochemical reactions and 814 metabolites was considered on the metabolic pathways. This system is comprised of almost all the metabolic pathways of E. coli, and the biomass composition of E. coli for constituting a biomass formation equation of the strain to be used as an object function in the metabolic flux analysis is as follows (Neidhardt et al., Escherichia coli and Salmonella: Cellular and Molecular Biology, 1996):

55% proteins, 20.5% RNA, 3.1% DNA, 9.1% lipids, 3.4% lipopolysaccharides, 2.5% peptidoglycan, 2.5% glycogen, 0.4% polyamines, 3.5% other metabolites, cofactors, and ions.

(B) Mannheimia

Mannheimia succiniciproducens MBEL55E (KCTC 0769BP), a strain whose entire genome has been decoded and functional analysis has been completed, is a Korean native strain isolated directly from the rumen of Korean native cattle, and has the ability to produce a large amount of succinic acid which can be used in various industrial fields.

It was found by a bioinformatics technique that the genome of Mannheimia consists of 2,314,078 bases (Hong et al., Nat. Biotechnol., 22:1275, 2004) and has 2,384 gene candidates. The genes of Mannheimia are distributed throughout the whole circular genome, and were classified according to their intracellular functions so that they were used to predict the characteristics of the entire genome.

By the entire analysis of genomic information, an in silico model of Mannheimia was constructed on a computer. A metabolic network was constructed with 373 enzymatic reaction equations and 352 metabolites, and on the basis of this result, a change in intracellular metabolic flux could be predicted.

### Example 2

### Target Gene Screening

The database of BioSilico (http://biosilico.kaist.ac.kr) in which the central metabolic pathways of succinic acid-overproducing Mannheimia and the central metabolic pathways of E. coli has been constructed was used to construct simulation models.

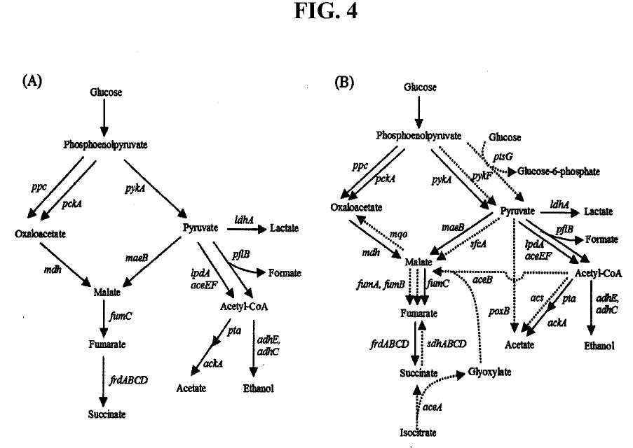

For the comparison of metabolisms, the metabolic pathway of Mannheimia (A), a succinic acid-overproducing strain, was compared to the metabolic pathway of E. coli (B), a target strain for producing succinic acid, and the results are shown in FIG. 4. Then, genes on the central metabolic pathways were compared, and genes were screened, which can be unnecessary for or interfere with the production of succinic acid in E. coli.

Genes on the central metabolic pathways for succinic acid production in Mannheimia were compared to genes on the central metabolic pathways for succinic acid production in E. coli, as a result, genes present only in E. coli were ptsG, pykF, pykA, mqo, sdhABCD, aceBA, poxB and acs. Among these genes, genes excluding poxB and acs known to be inoperable in anaerobic conditions were first screened as candidate genes which can be unnecessary for or interfere with the production of succinic acid. Namely, ptsG, pykF, pykA, mqo, sdhABCD and aceBA were screened.

### Example 3

### Screening of Mutant Strains

To produce specific metabolites using microorganisms, the specific growth rate of cells should be generally considered in addition to production yield. Generally, strains seem to grow to maximize cellular components but not to grow to form useful products, and this growth is expressed as specific growth rate. Accordingly, to predict which gene deletions make useful products maximal while making specific growth rate excellent, the metabolic flux analysis technology was used.

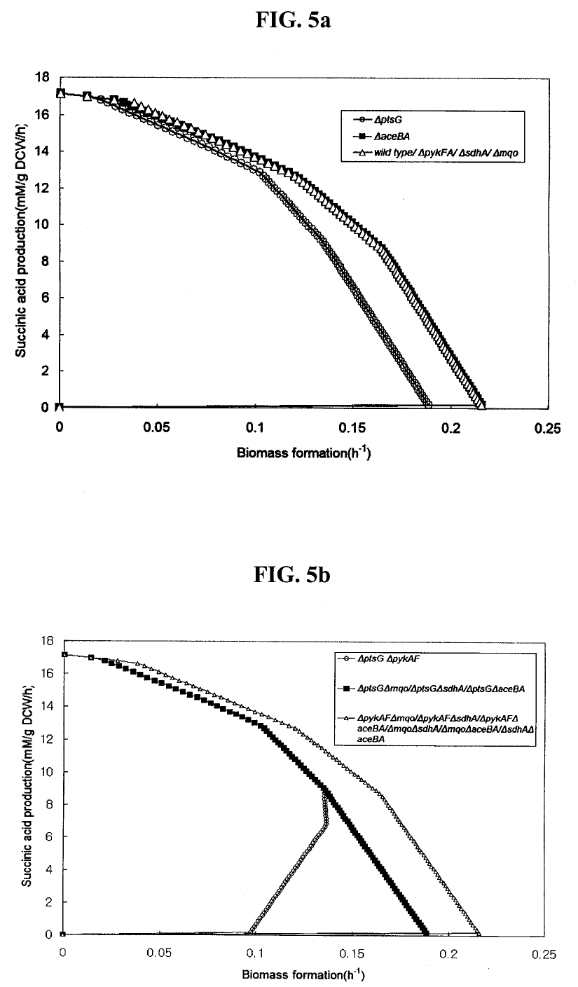

To simultaneously consider production yield and specific growth rate resulting from one gene deletion and two combinations of gene deletions for the first-considered candidate genes, two objective functions (i.e., specific growth rate and the formation rate of useful products) were selected and plotted on x- and y-axes, respectively, and the results are shown in FIG. 5a and FIG. 5b. Namely, a curve allowing the optimal product yield versus the specific growth rate of the strain to be obtained was selected, thus selecting a combination of genes corresponding to the target metabolic pathways.

(A) Simulation of Multi-Gene Mutant Strains

To construct a multi-gene mutant for each gene combination, numerous combinations of mutations should be made. Making such numerous mutations by actual experiments is actually very difficult or next to impossible. Thus, in silico simulation was performed on the basis of a simulation system for determining the trade-off curves of product formation rate versus specific growth rate, constructed for each of mutations. The simulation was conducted using MetaFluxNet 1.6® which can be downloaded from the website “http://mbel.kaist.ac.kr” (Lee et al., Bioinformatics, 19:2144, 2003).

In the simulation, glucose was used as a carbon source, and oxygen intake rate was set to zero in order to consider a generally known glucose intake rate of 10 mmol/g DCW/h and anaerobic conditions. Also, the biochemical reaction rate corresponding to the considered gene deletions was set to zero.

To plot the trade-off curves, the algorithm suggested in the prior literature was modified (Burgard et al., Biotechnol. Bioeng., 84:647, 2003). In this prior literature, a method for finding candidate genes by trade-off curves is not exactly described, whereas, in the method used in the present invention, a combination of candidate genes, which has a curve showing no reduction in biomass even in the case of a reduction in the production rate of a useful substance, could be selected by examining the relation between the useful substance production rate and biomass formation rate of a mutant strain with deletions of relevant genes thus, the abilities of relevant mutant strains to produce a useful substance could be compared.

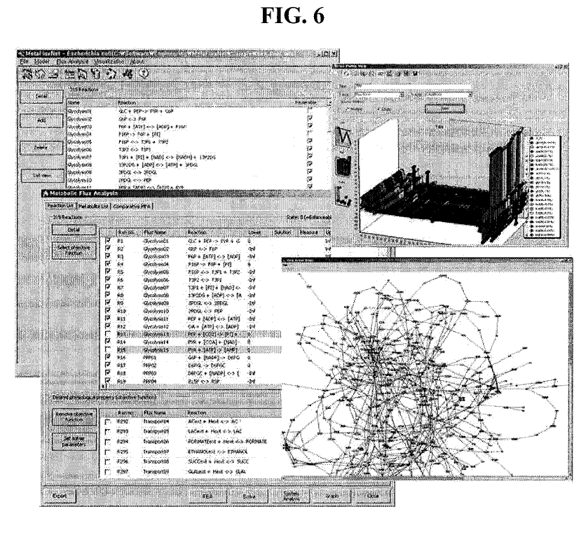

Namely, the maximized value of useful product formation rate and the minimized value of useful product formation rate were first calculated to determine the allowable range of useful product formation rate, and then, specific growth rate was maximized within the allowable range, thus plotting the trade-off curve between the two objective functions. FIG. 6 shows an example of a trade-off curve plotted using MetaFluxNet.

To examine the yield of a useful product, considering specific growth rate, the trade-off curve between product formation rate and specific growth rate necessary for the application of the metabolic flux control technology was determined (FIGS. 5a and 5b).

As shown in FIG. 5b, the results of the examination of gene combinations corresponding to the target metabolic pathways showed that a curve capable of obtaining the optimum production yield versus specific growth rate was obtained in the case of a mutant strain with simultaneous deletions of ptsG, pykF and pykA, unlike strains with combinations of deletions of other genes. Namely, in the case of the relevant genes, a curve, different from the tendency of an increase in specific growth rate as useful substance production rate decreases, could be obtained, in which case succinic acid production rate was also the most excellent.

The numerical examination of such results showed that if E. coli with simultaneous deletions of ptsG, pykF and pykA was cultured in anaerobic conditions, succinic acid was overproduced as compared to the cases of wild-type strains and strains with deletions of combinations of other genes (Table 1).

TABLE 1
Simulation results for each of mutants

| | | | Maximum |
|---|---|---|---|
| | | Succinic acid | production |
| Deletion of | Maximum biomass | production rate | capacity of |
| genes | formation rate (h⁻¹) | (mmol/DCW/h) | succinic acid^{a} |
| Wild type | 0.2156 | 0.1714 | 1.000 |
| pykFA | 0.2156 | 0.1714 | 1.000 |
| ptsG | 0.1884 | 0.1714 | 0.8738 |
| ptsG pykFA | 0.1366 | 6.834 | 25.26 |
| ptsG mqo | 0.1884 | 0.1714 | 0.8738 |
| ptsG sdhA | 0.1884 | 0.1714 | 0.8738 |
| ptsG aceBA | 0.1884 | 0.1714 | 0.8738 |
| pykFA sdhA | 0.2156 | 0.1714 | 1.000 |
| pykFA aceBA | 0.2156 | 0.1714 | 1.000 |
| mqo sdhA | 0.2156 | 0.1714 | 1.000 |
| mqo aceBA | 0.2156 | 0.1714 | 1.000 |
| sdhA aceBA | 0.2156 | 0.1714 | 1.000 |

^{a}Calculation formula: (succinic acid production rate of mutant × maximum biomass formation rate)/(succinic acid production rate of wild type × maximum biomass formation rate)

B. Actual Test Results

To construct E. coli mutant strains on the basis of the simulation results, a standard protocol for DNA engineering was used and red recombinase present in the red operon of lambda bacteriophage was used (Sambrook et al., Molecular Cloning: a Laboratory Manual, 3rd edition, 2001; Datsenko et al., Proc. Natl. Acad. Sci. USA, 97:6640, 2000). First, a DNA template containing an antibiotic-resistant gene was subjected to two-step PCR using primers (see Table 2) containing oligonucleotide located upstream and downstream of the target gene.

TABLE 2

| | PCR | | |
|---|---|---|---|
| Templates | steps | Primers | Sequences (5′-3′) |
| SpR | 1^{st} | SEQ ID NO: 1: | TGC CCG CCG TTG TAT CGC ATG TTA TGG CAG |
| | | PTSG1 | GGG GAT CGA TCC TCT AGA |
| | | SEQ ID NO: 2: | TGC AGC AAC CAG AGC CGG TGC CAT TTC GCT |
| | | PTSG2 | GGG CCG ACA GGC TTT |
| | 2^{nd} | SEQ ID NO: 3: | TGG GCG TCG GTT CCG CGA ATT TCA GCT GGC |
| | | PTSG3 | TGC CCG CCG TTG TAT CGC |
| | | SEQ ID NO: 4: | GAG GTT AGT AAT GTT TTC TTT ACC ACC AAA |
| | | PTSG4 | TGC AGC AAC CAG AGC CGG |
| TcR | 1^{st} | SEQ ID NO: 5: | TGG ACG CTG GCA TGA ACG TTA TGC GTC TGA |
| | | PYKF1 | GGG TAG ATT TCA GTG CAA |
| | | SEQ ID NO: 6: | CGC CTT TGC TCA GTA CCA ACT GAT GAG CCG |
| | | PYKF2 | GGG TTC CAT TCA GGT CGA |
| | 2^{nd} | SEQ ID NO: 7: | CCG AAT CTG AAG AGA TGT TAG CTA AAA TGC |
| | | PYKF3 | TGG ACG CTG GCA TGA ACG |
| | | SEQ ID NO: 8: | AAG TGA TCT CTT TAA CAA GCT GCG GCA CAA |
| | | PYKF4 | CGC CTT TGC TCA GTA CCA |
| CmR | 1^{st} | SEQ ID NO: 9: | GGC ATA CCA TGC CGG ATG TGG CGT ATC ATT |
| | | MQO1 | GGG GTT TAA GGG CAC CAA |
| | | SEQ ID NO: 10: | GAA CTA CGG CGA GAT CAC CCG CCA GTT AAT |
| | | MQO2 | GCC CCG GGC TTT GCG CCG |
| | 2^{nd} | SEQ ID NO: 11: | TGG CGC GTC TTA TCA GCA TAC GCC ACA TCC |
| | | MQO3 | GGC ATA CCA TGC CGG ATG |
| | | SEQ ID NO: 12: | AGC CAC GCG TAC GGA AAT TGG TAC CGA TGT |
| | | MQO4 | GAA CTA CGG CGA GAT CAC |
| KmR | 1^{st} | SEQ ID NO: 13: | CAG TCA GAG AAT TTG ATG CAG TTG TGA TTG |
| | | SDH1 | ATC GGG GGG GGG GGA AAG |
| | | SEQ ID NO: 14: | ATC GGC TCT TTC ACC GGA TCG ACG TGA GCG |
| | | SDH2 | ATC CCA ATT CTG ATT AGA |
| | 2^{nd} | SEQ ID NO: 15: | GTT GTG GTG TGG GGT GTG TGA TGA AAT TGC |
| | | SDH3 | CAG TCA GAG AAT TTG ATG |
| | | SEQ ID NO: 16: | ATC ATG TAG TGA CAG GTT GGG ATA ACC GGA |
| | | SDH4 | ATC GGC TCT TTC ACC GGA |
| PmR | 1^{st} | SEQ ID NO: 17: | GCA CCT TGT GAT GGT GAA CGC ACC GAA GAA |
| | | ACEBA1 | CGA GCT CGG TAC CCG GGC |
| | | SEQ ID NO: 18: | CTT TCG CCT GTT GCA GCG CCT GAC CGC CAG |
| | | ACEBA2 | CAA TAG ACC AGT TGC AAT |
| | 2^{nd} | SEQ ID NO: 19: | GAC GCG CCG ATT ACT GCC GAT CAG CTG CTG |
| | | ACEBA3 | GCA CCT TGT GAT GGT GAA |
| | | SEQ ID NO: 20: | ATC CCG ACA GAT AGA CTG CTT CAA TAC CCG |
| | | ACEBA4 | CTT TCG CCT GTT GCA GCG |
| KmR | 1^{st} | SEQ ID NO: 21: | CAC CTG GTT GTT TCA GTC AAC GGA GTA TTA |
| | | PYKA1 | CAT CGG GGG GGG GGG AAA G |
| | | SEQ ID NO: 22: | GTG GCG TTT TCG CCG CAT CCG GCA ACG TAC |
| | | PYKA2 | ATC CCA ATT CTG ATT AG |
| | 2^{nd} | SEQ ID NO: 23: | TTA TTT CAT TCG GAT TTC ATG TTC AAG CAA |
| | | PYKA3 | CAC CTG GTT GTT TCA GTC |
| | | SEQ ID NO: 24: | GTT GAA CTA TCA TTG AAC TGT AGG CCG GAT |
| | | PYKA4 | GTG GCG TTT TCG CCG CAT C |

The PCR product was transformed into a parent strain, and the target gene was replaced with the antibiotic-resistant gene by double homologous recombination, thus constructing mutant strains with a deletion of the target gene. The constructed strains are shown in Table 3.

TABLE 3

| Strains | Characteristics |
|---|---|
| E. coli W3110 | Coli Genetic Stock Center strain No. 4474 |
| E. coli W3110G | ptsG::Sp^{r} |
| E. coli W3110GF | ptsG::Sp^{r}, pykF::Tc^{r} |
| E. coli W3110GFA | ptsG::Sp^{r}, pykF::Tc^{r}, pykA::Km^{r} |
| E. coli W3110GFO | ptsG::Sp^{r}, pykF::Tc^{r}, mqo::Cm^{r} |
| E. coli W3110GFH | ptsG::Sp^{r}, pykF::Tc^{r}, sdh::Km^{r} |
| E. coli W3110GFHO | ptsG::Sp^{r}, pykF::Tc^{r}, sdh::Km^{r}, mqo::Cm^{r} |
| E. coli W3110GFHOE | ptsG::Sp^{r}, pykF::Tc^{r}, sdh::Km^{r}, mqo::Cm^{r}, |
| | aceBA::Pm^{r} |

In Table 3, Sp^{r} represents spectinomycin resistance, Tc^{r} represents tetracycline resistance, Cm^{r} represents chloramphenicol resistance, Km^{r} represents kanamycin resistance, and Pm^{r} represents phleomycin resistance.

Each of the mutants constructed as described above was cultured at an initial glucose concentration of 60 mM in anaerobic conditions for 24 hours, and examined for the concentration of remaining glucose and the concentrations of succinic acid, lactate, formate, acetate and ethanol. The results are shown in Table 4 (ratio of each of organic acids in actual test results). As can be seen in Table 4, in the case of the mutant strain (W3110GFA) with deletions of ptsG, pykF and pykA, the succinic acid ratio versus other organic acids (S/A ratio) increased 8.28 times, compared to the wild-type strain (W3110).

TABLE 4
Concentrations of succinic acid, lactate, formate, acetate and ethanol in actual test

| | | Concentration of fermentation substrate or products (mM)^{a} | | | | | | Succinic | |
|---|---|---|---|---|---|---|---|---|---|
| | | | succinic | | | | | acid | [symbol] |
| Strains | OD₆₀₀ | glucose^{b} | acid | lactate | formate | acetate | ethanol | ratio^{c} | Fold^{d} |
| W3110 | 1.79 ± 0.11 | 5.07 ± 0.45 | 2.43 ± 0.03 | 10.62 ± 2.42 | 88.03 ± 0.42 | 40.10 ± 0.20 | 5.77 ± 0.06 | 0.017 | 1 |
| W3110G | 1.47 ± 0.01 | 5.66 ± 1.77 | 2.16 ± 0.08 | 13.71 ± 3.46 | 88.57 ± 2.97 | 40.69 ± 0.97 | 5.98 ± 0.09 | 0.014 | 0.86 |
| W3110GF | 1.46 ± 0.04 | 4.28 ± 1.42 | 2.83 ± 0.07 | 14.13 ± 1.32 | 86.25 ± 2.02 | 40.10 ± 0.58 | 5.92 ± 0.24 | 0.019 | 1.15 |
| W3110GFA | 0.73 ± 0.06 | 20.57 ± 3.02 | 8.16 ± 0.01 | 5.47 ± 0.49 | 27.47 ± 2.94 | 16.48 ± 1.55 | 1.88 ± 0.24 | 0.137 | 8.29 |
| W3110GFO | 1.49 ± 0.07 | 4.68 ± 0.35 | 2.67 ± 0.33 | 12.97 ± 0.06 | 88.39 ± 0.81 | 40.89 ± 0.18 | 6.00 ± 0.08 | 0.018 | 1.07 |
| W3110GFH | 1.35 ± 0.01 | 4.70 ± 0.39 | 2.51 ± 0.02 | 15.18 ± 1.49 | 85.86 ± 0.10 | 38.88 ± 0.16 | 5.84 ± 0.06 | 0.017 | 1.02 |
| W3110GFHO | 1.28 ± 0.11 | 4.82 ± 0.48 | 2.58 ± 0.03 | 17.06 ± 0.45 | 90.40 ± 2.55 | 40.40 ± 0.49 | 6.20 ± 0.06 | 0.016 | 1 |
| W3110GFOHE | 1.27 ± 0.05 | 4.25 ± 0.27 | 2.49 ± 0.18 | 13.31 ± 0.78 | 85.98 ± 0.38 | 38.66 ± 0.02 | 5.84 ± 0.01 | 0.017 | 1.03 |

^{a}24 hour anaerobic culture
^{b}Remaining glucose concentration (initial glucose concentration: 50 mM).
^{c}Calculation formula: (succinic acid)/(succinic acid + lactate + formate + acetate + ethanol).
^{d}Calculation formula: Succinic acid ratio/0.017(Succinic acid ratio of wild type).

From the above results, it can be seen that the present invention can provide the metabolic and genetic engineering approach comprising comparatively analyzing the genomic information of E. coli, a typical target strain for the production of a useful substance and the genomic information of the Mannheimia strain overproducing succinic acid, and using a simulation program to improve the E. coli strain into a mutant strain producing a large amount of succinic acid.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof. Those skilled in the art will appreciate that simple modifications, variations and additions to the present invention are possible, without departing from the scope and spirit of the invention as disclosed in the accompanying claims.

### INDUSTRIAL APPLICABILITY

As can be seen from the foregoing, according to the present invention, an improved strain can be effectively constructed by the metabolic and genetic engineering approach comprising comparatively analyzing the genomic information of a target strain for producing a useful substance and the genomic information of a strain producing a large amount of the useful substance so as to screen candidate genes and performing in silico simulation on the screened candidate genes to select a combination of genes to be deleted, which shows an improvement in the production of the useful substance. Accordingly, the time, effort and cost required for an actual wet test can be significantly reduced.

1. A method for improving a useful substance-producing strain, the method comprising the steps of:
(a) selecting a target strain for producing a useful substance and a useful substance-overproducing strain, and constructing metabolic flux analysis model systems for the two strains;
(b) screening genes absent in the useful substance-overproducing strain among genes which are present in the useful substance-producing target strain and are unnecessary for or interfere with the growth of cells;
(c) constructing combinations of genes to be deleted, from the screened genes;
(d) performing in silico simulation on a mutant strain obtained by deleting each of the combinations of genes constructed in the step (c), from the target strain for producing a useful substance, using the metabolic flux analysis model systems constructed in the step (a);
(e) selecting a combination of genes to be deleted, which is excellent in useful substance production yield versus specific growth rate, from the simulation results; and
(f) constructing a mutant strain with a deletion of the selected combination of genes.

2. The method for improving a useful substance-producing strain according to claim 1, wherein the in silico simulation is performed by plotting a trade-off curve between product formation rate and specific growth rate and comparing the specific growth rate of the mutant strain to the yield of the useful substance.

3. The method for improving a useful substance-producing strain according to claim 1, wherein the a target strain for producing a useful substance is E. coli.

4. The method for improving a useful substance-producing strain according to claim 1, wherein the method additionally comprises the step of: (g) culturing the constructed mutant strain to experimentally examine the useful substance production of the mutant strain.

5. A method for improving a succinic acid-producing strain, the method comprising the steps of:
(a) selecting a target strain for producing succinic acid and a succinic acid-overproducing strain and constructing metabolic flux analysis model systems for the two strains;
(b) screening genes absent in the succinic acid-overproducing strain among genes which are present in the target strain for producing succinic acid and are unnecessary for or interfere with the growth of cells;
(c) constructing combinations of genes to be deleted from the screened genes;
(d) performing in silico simulation on a mutant strain obtained by deleting each of the combinations of genes constructed in the step (c), from the target strain for producing succinic acid, using the metabolic flux analysis model systems constructed in the step (a);
(e) selecting a combination of genes to be deleted, which is excellent in succinic acid production yield versus specific growth rate, from the simulation results; and
(f) constructing a mutant strain with a deletion of the selected combination of genes.

6. The method for improving a succinic acid-producing strain according to claim 5, wherein the in silico simulation is performed by plotting a trade-off curve between succinic acid formation rate and specific growth rate and comparing the specific growth rate of the mutant strain to the yield of succinic acid.

7. The method for improving a succinic acid-producing strain according to claim 5, wherein the succinic acid-overproducing strain is the genus Mannheimia.

8. The method for improving a succinic acid-producing strain according to claim 7, wherein the succinic acid-overproducing strain is Mannheimia succiniciproducens MBEL55E (KCTC 0769BP).

9. The method for improving a succinic acid-producing strain according to claim 5, wherein the target strain for producing succinic acid is E. coli.

10. The method for improving a succinic acid-producing strain according to claim 5, wherein the gene screened in the step (b) is selected from the group consisting of ptsG, pykF, pykA, mqo, sdhA, sdhB, sdhC, sdhD, aceB and aceA.

11. The method for improving a succinic acid-producing strain according to claim 5, wherein the combination of genes to be deleted, which is selected in the step (e), consists of ptsG, pykF and pykA.

12. The method for improving a succinic acid-producing strain according to claim 5, wherein the method additionally comprises the step of: (g) culturing the constructed mutant strain to experimentally examine the succinic acid production of the mutant strain.

13. A mutant strain with deletions of ptsG, pykF and pykA genes, and having the ability to produce high yield of succinic acid.

14. A method for producing succinic acid, the method comprises culturing the mutant strain of claim 13 in anaerobic conditions.

15. A mutant of E. coli with deletions of ptsG, pykF and pykA genes.

16. A method for producing succinic acid, the method comprises culturing the mutant of E. coli according to claim 15 in anaerobic conditions.
